(19) United States
(12) Patent Application Publication
(10) Pub. No.: US 2010/0036434 A1
(43) Pub. Date: Feb. 11, 2010

(54) RESCUE REDUCTION BONE ANCHOR

(75) Inventor: Kameron Scott Ely, Cedar Park, TX (US)

Correspondence Address: SPRINKLE IP LAW GROUP, 1301 W. 25TH STREET, SUITE 408, AUSTIN, TX, 78705, US

(73) Assignee: Abbott Spine Inc., Austin, TX (US)

(21) Appl. No.: 12/186,393

(22) Filed: Aug. 5, 2008

(51) Int. Cl. A61B 17/04

(52) U.S. Cl. 606/305; 606/301; 606/308

(57) ABSTRACT

Systems and methods for reducing spinal stabilization rods. One embodiment provides an instrument including a body (defining a threaded slot for receiving the spinal stabilization rod), an alignment feature, and a flange (for mounting the instrument on a bone anchor), each of which can correspond to portions of the bone anchor. The alignment feature can align the threads of the instrument and bone anchor to form substantially continuous threads. The alignment features can be a pin and a hole oriented radially with respect to the instrument. An actuator can actuate the instrument alignment features toward, or away from, the instrument body. An actuator slot can correspond to the instrument slot. The threads of the instrument slot can include a thread diameter transition portion. In some embodiments, the instrument threads can include an anti-splay feature which mitigates reaction moments arising from the reduction of the spinal stabilization rod.

### TECHNICAL FIELD

This disclosure relates generally to spinal implants, and more particularly to spinal implants and methods for reducing and attaching spinal stabilization rods to boney structures (such as vertebrae) when the spinal stabilization rods may be proud of their intended position by some distance.

### BACKGROUND

The human spine consists of segments known as vertebrae separated by intervertebral disks and held together by various ligaments. There are 24 movable vertebrae—7 cervical, 12 thoracic, and 5 lumbar. Each of the movable vertebra has a somewhat cylindrical bony body (often referred to as the centrum), a number of winglike projections, and a bony arch. The bodies of the vertebrae form the supporting column of the skeleton. The arches of the vertebrae are positioned so that the spaces they enclose form a curvilinear passage which is often referred to as the vertebral canal. The vertebral canal houses and protects the spinal cord (which includes bundles of sensory and motor nerves for sensing conditions in or affecting the body and commanding movements of various muscles). Within the vertebral canal, spinal fluid can circulate to cushion the spinal cord and carry immunological cells to it, thereby protecting the sensory and motors nerves therein from mechanical damage and disease. Ligaments and muscles are attached to various projections of the vertebrae such as the superior-inferior, transverse, and spinal processes. Other projections, such as vertebral facets, join adjacent vertebrae to each other, in conjunction with various attached muscles, tendons, etc. while still allowing the vertebrae to move relative to each other.

Spines may be subject to abnormal curvature, injury, infections, tumor formation, arthritic disorders, punctures of the intervertebral disks, slippage of the intervertebral disks from between the vertebrae, or combinations thereof. Injury or illness, such as spinal stenosis and prolapsed disks may result in intervertebral disks having a reduced disk height, which may lead to pain, loss of functionality, reduced range of motion, disfigurement, and the like. Scoliosis is one relatively common disease which affects the spinal column. It involves moderate to severe lateral curvature of the spine and, if not treated, may lead to serious deformities later in life. Such deformities can cause discomfort and pain to the person affected by the deformity. In some cases, various deformities can interfere with normal bodily functions. For instance, some spinal deformities can cause the affected person's rib cage to interfere with movements of the respiratory diaphragm, thereby making respiration difficult. Additionally, some spinal deformities noticeably alter the posture, gate, appearance, etc. of the affected person, thereby causing both discomfort and embarrassment to those so affected. One treatment involves surgically implanting devices to correct such deformities, to prevent further degradation, and to mitigate symptoms associated with the conditions which may be affecting the spine.

Modern spine surgery often involves spinal stabilization through the use of spinal implants or stabilization systems to correct or treat various spine disorders and/or to support the spine. Spinal implants may help, for example, to stabilize the spine, correct deformities of the spine, facilitate fusion of vertebrae, or treat spinal fractures and other spinal injuries. Spinal implants can alleviate much of the discomfort, pain, physiological difficulties, embarrassment, etc. that may be associated with spinal deformities, diseases, injury, etc.

Spinal stabilization systems typically include corrective spinal instrumentation that is attached to selected vertebra of the spine by bone anchors, screws, hooks, clamps, and other implants hereinafter referred to as “bone anchors.” Some corrective spinal instrumentation includes spinal stabilization rods, spinal stabilization plates that are generally parallel to the patients back, or combinations thereof In some situations, corrective spinal instrumentation may also include superior-inferior connecting rods that extend between bone anchors (or other attachment instrumentation) attached to various vertebrae along the affected portion of the spine and, in some situations, adjacent vertebrae or adjacent boney structures (for instance, the occipital bone of the cranium or the coccyx). Spinal stabilization systems can be used to correct problems in the cervical, thoracic, and lumbar portions of the spine, and are often installed posterior to the spine on opposite sides of the spinous process and adjacent to the superior-inferior process. Some implants can be implanted anterior to the spine and some implants can be implanted at other locations as selected by surgical personnel such as at posterior locations on the vertebra.

Often, spinal stabilization may include rigid support for the affected regions of the spine. Such systems can limit movement in the affected regions in virtually all directions. Such spinal stabilizations are often referred to as “static” stabilization systems and can be used in conjunction with techniques intended to promote fusion of adjacent vertebrae in which the boney tissue of the vertebrae grow together, merge, and assist with immobilizing one or more intervertebral joints. More recently, so called “dynamic” spinal stabilization systems have been introduced wherein the implants allow at least some movement (e.g., flexion or extension) of the affected regions of the spine in at least some directions. Dynamic stabilization systems therefore allow the patient greater freedom of motion at the treated intervertebral joint(s) and, in some cases, improved quality of life over that offered by static stabilization systems.

### SUMMARY

One embodiment provides an instrument for reducing a spinal stabilization rod into a bone anchor using a closure member. The rod reduction instrument can include a body, an alignment feature, and a flange. The instrument alignment feature can correspond to the bone anchor alignment feature. The instrument flange can be at the distal end of the rod reduction instrument and can be adapted for mounting the rod reduction instrument on the bone anchor. The instrument body can define a threaded slot which extends between the proximal end of the rod reduction instrument and the instrument flange at the distal end of the rod reduction instrument. The instrument slot can correspond to a slot of the bone anchor. The instrument alignment features and the bone anchor alignment features can align the threads of the instrument slot and the threads of the bone anchor to form substantially continuous threads when the rod reduction instrument is mounted on the bone anchor. The instrument alignment features and the bone anchor alignment features can be, respectively, a pin and a corresponding hole oriented in a radial direction relative to the longitudinal axis of the rod reduction instrument.

In some embodiments, the rod reduction instrument can include an alignment feature actuator coupled to the instrument alignment feature to actuate the instrument alignment feature by pivoting about an actuator gusset of the instrument. The alignment feature actuator can bias the instrument alignment feature radially toward the bone anchor alignment feature when the rod reduction instrument is mounted on the bone anchor. The alignment feature actuator can define a slot corresponding to the instrument slot. In some embodiments, the instrument slot can include a thread transition portion across which the instrument threads transition from one diameter to another diameter. The instrument threads can include an anti-splay feature to prevent the instrument slot walls from splaying when the spinal stabilization rod is reduced through the instrument slot.

One embodiment provides a method of reducing a spinal stabilization rod into a bone anchor. The spinal stabilization rod reduction method can include receiving the spinal stabilization rod in a slot of a rod reduction instrument. The closure member may also be received in the instrument slot. The method of reducing a spinal stabilization rod can further include mounting the rod reduction instrument to the bone anchor. Threads of the instrument and threads of the bone anchor can be aligned with each other so that the instrument threads and the bone anchor threads form substantially continuous threads along the instrument and bone anchor slots. The closure member may be driven along the substantially continuous threads to urge the spinal stabilization rod into the bone anchor. While the closure member is being driven, splaying of the instrument slot walls can be prevented.

In some embodiments, the method of reducing a spinal stabilization rod can include driving the closure member along a thread transition portion of the instrument threads. Across the thread transition portion, the instrument threads can transition from one diameter to another diameter. Aligning the instrument threads and the bone anchor threads can include actuating an alignment feature actuator of the instrument to urge an alignment feature of the instrument away from the bone anchor and can include receiving the spinal stabilization rod in a slot of the actuator (which can correspond to the instrument slot). The method of reducing a spinal stabilization rod can include allowing the alignment feature actuator to urge the instrument alignment features toward alignment features of the bone anchor. Actuation of the alignment feature actuator can include pivoting the alignment feature actuator to cause a portion of the alignment feature actuator to move in a radial direction relative to the rod reduction instrument.

One embodiment provides a kit for reducing a spinal stabilization rod using a closure member. The rod reduction kit can include a bone anchor and a rod reduction instrument. The bone anchor can define a slot for receiving the spinal stabilization rod and the closure member. The bone anchor can include an alignment feature and a wall of the bone anchor slot. The wall of the bone anchor slot can define threads. The rod reduction instrument can include a body, another alignment feature, and a flange. The instrument alignment feature can correspond to the bone anchor alignment feature. The instrument flange can be at the distal end of the rod reduction instrument and can be adapted for mounting the rod reduction instrument on the bone anchor. The rod reduction instrument can define another slot which extends between the proximal end of the rod reduction instrument and the instrument flange at the distal end of the rod reduction instrument. The instrument and bone anchor slots can correspond to each other.

The instrument slot wall can include threads along a portion of the instrument slot wall and can include an anti-splay feature and a thread transition portion. The instrument alignment features and the bone anchor alignment features can align the instrument threads and bone anchor threads to form substantially continuous threads when the rod reduction instrument is mounted on the bone anchor. The instrument and bone anchor alignment features can be, respectively, a pin and a hole oriented in a radial direction relative to the longitudinal axis of the instrument. The instrument alignment features can be urged toward the bone anchor alignment features by an alignment feature actuator.

In some embodiments, an alignment feature actuator can be a sleeve slidably engaging a member coupled to the instrument alignment feature. The sleeve can lock the instrument alignment feature in a position wherein the instrument alignment feature is engaged with an anchor alignment feature by holding the member against the body of the instrument.

Embodiments disclosed herein provide many advantages. For example, some embodiments provide rod reduction instruments and methods of reducing spinal stabilization rods in which closure members can be driven across the transition from threads of the rod reduction instrument to threads of a bone anchor substantially without binding or loosing mechanical advantage. Some embodiments provide rod reduction instruments and methods of reducing spinal stabilization rods which can align the instrument and bone anchor threads without disturbing tissues which might be proximal to the rod reduction instrument and bone anchor. Some embodiments allow surgical personnel to actuate alignment features of the rod reduction instrument by grasping an ergonomically shaped and dimensioned portion of the rod reduction instrument and alignment feature actuators thereof. Some embodiments allow surgical personnel to advance a closure member relatively rapidly through the body of a rod reduction instrument. Some embodiments allow surgical personnel to drive the closure member against the spinal stabilization rod despite reactions of the spinal stabilization rod arising from its reduction into a bone anchor.

These, and other, aspects will be better appreciated and understood when considered in conjunction with the following description and the accompanying drawings. The following description, while indicating various embodiments and numerous specific details thereof, is given by way of illustration and not of limitation. Many substitutions, modifications, additions or rearrangements may be made within the scope of the disclosure, and the disclosure includes all such substitutions, modifications, additions or rearrangements.

### BRIEF DESCRIPTION OF THE DRAWINGS

A more complete understanding of the present disclosure and the advantages thereof may be acquired by referring to the following description, taken in conjunction with the accompanying drawings in which like reference numbers indicate like features and wherein:

FIG. 1 depicts a human axial skeleton;

FIG. 2 depicts one embodiment of a spinal stabilization system;

FIG. 3 depicts one embodiment of an instrument being used in conjunction with a bone anchor to reduce a spinal stabilization rod into the bone anchor;

FIG. 4 depicts a cross sectional view of one embodiment of an instrument;

FIG. 5 depicts a perspective view of one embodiment of an instrument and one embodiment of a bone anchor;

FIG. 6 depicts a side elevation view of one embodiment of an instrument;

FIG. 7 depicts a side elevation of one embodiment of an instrument;

FIG. 8 depicts one embodiment of a method for reducing a spinal stabilization rod into a bone anchor; and

FIGS. 9A-9D depict cross sectional views of one embodiment of an instrument and one embodiment of a bone anchor.

### DETAILED DESCRIPTION

The disclosure and the various features and advantageous details thereof are explained more fully with reference to the non-limiting embodiments detailed in the following description. Descriptions of well known starting materials, manufacturing techniques, components, and equipment are omitted so as not to unnecessarily obscure the disclosure in detail. Skilled artisans should understand, however, that the detailed description and the specific examples, while disclosing preferred embodiments of the disclosure, are given by way of illustration only and not by way of limitation. Various substitutions, modifications, and additions within the scope of the underlying inventive concept(s) will become apparent to those skilled in the art after reading this disclosure. Skilled artisans can also appreciate that the drawings disclosed herein are not necessarily drawn to scale.

As used herein, the terms “comprises,” “comprising,” “includes,” “including,” “has,” “having” or any other variation thereof, are intended to cover a non-exclusive inclusion. For example, a process, process, article, or apparatus that comprises a list of elements is not necessarily limited only those elements but may include other elements not expressly listed or inherent to such process, process, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Additionally, any examples or illustrations given herein are not to be regarded in any way as restrictions on, limits to, or express definitions of, any term or terms with which they are utilized. Instead, these examples or illustrations are to be regarded as being described with respect to one particular embodiment and as illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example”, “for instance”, “e.g.”, “in one embodiment”.

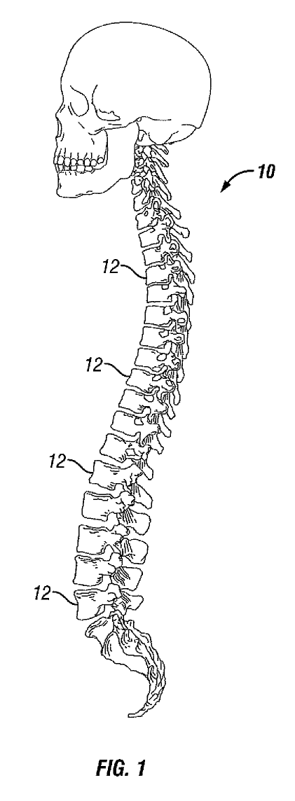

FIG. 1 depicts a human axial skeleton including a skull (composed of numerous cranial bones (such as parietal bones, temporal bones, zygomatic bones, mastoid bones, maxilla bones, mandible bones, etc.) and spine 10 including numerous vertebrae 12 and intervertebral disks separating certain vertebrae 12. Ligaments can join vertebrae 12 and muscles and tendons can attach to vertebrae 12. As discussed previously, spine 10 carries loads imposed on the patient's body and loads generated by the patient. Vertebrae 12 cooperate to allow spine 10 to extend, flex, rotate, etc. under the influence of various muscles, tendons, ligaments, etc. attached to spine 10. Spine 10 can also cooperate with various muscles, tendons, ligaments, etc. to cause other anatomical features of the patient's body to move and exert force on objects in the environment.

However, certain conditions can cause damage to spine 10, vertebrae 12, certain intervertebral discs, etc. and can impede the ability of spine 10 to move in various manners. These conditions include, but are not limited to abnormal curvature, injury, infections, tumor formation, arthritic disorders, puncture, or slippage of the intervertebral disks, and injuries or illness such as spinal stenosis and prolapsed disks. As some of these conditions progress, come into existence, or persist, various symptoms can indicate the desirability of stabilizing spine 10 or curing deformities thereof. As a result of these various conditions, the ability of the patient to move, with or without pain or discomfort, can be impeded. Additionally, certain physiological processes such as breathing can be impeded by such conditions. Based on various indications, medical personnel might recommend attaching one or more spinal stabilization or correction systems (hereinafter spinal stabilization systems) to vertebra 12 of spine 10 (among other possible remedial actions such as physical therapy) to correct the particular condition(s) from which the patient may be suffering and to prevent further deterioration of spine 10, vertebra 12, etc.

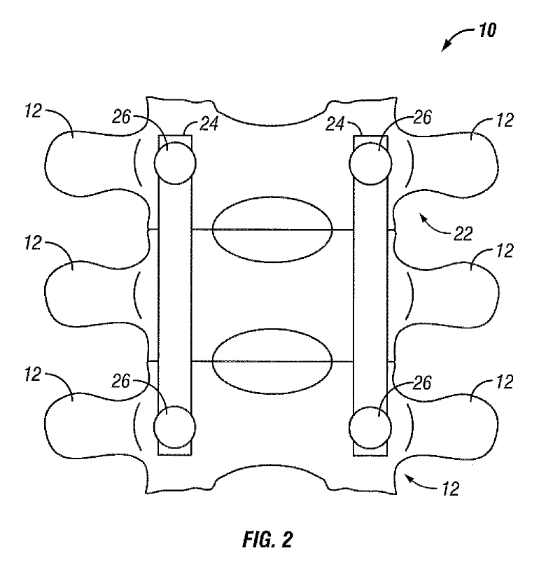

FIG. 2 depicts a top plan view of a portion of spine 10 including various vertebrae 12, inter-vertebral disks, spinous processes, vertebral facets, intravertebral area. etc. For clarity; FIG. 2 omits the muscles, tendons, ligaments, etc. that might be associated with spine 10. FIG. 2 also depicts spinal stabilization system 22 which can include a pair of spinal stabilization rods 24 and bone fastener assemblies 26 which can attach spinal stabilization rods 24 to vertebra 12 or other boney structures. One particular spinal stabilization rod 24 and its associated bone fastener assemblies 26 can be attached to vertebrae 12 on one side of a particular spinous process while the other spinal stabilization rod 24 can be attached to vertebrae 12 on the other side of the spinous process. Spinal stabilization rods 24 can be static (allowing little or no relative motion between adjacent vertebrae 12 of spine 10) or dynamic (allowing at least some relative motion between adjacent vertebrae 12 of spine 10). As will be discussed with more particularity herein, bone anchors of various embodiments can be used to attach spinal stabilization system 22 to vertebrae 12 as well as other boney structures. For instance, potential attachment points for spinal stabilization system 22 can include superior-inferior processes, transverse processes, vertebral facets, various surfaces exposed by surgical personnel removing portions of vertebrae 12, etc. Spinous processes and superior-inferior processes allow tendons, muscles, etc. to attach to spine 10 for movement of spine 10 and various anatomical structures which are attached to spine 10. These anatomical structures can include the patient's ribs, hips, shoulders, head, legs, etc. Spinous processes extend generally in a posterior and slightly inferior direction from vertebrae 12. Superior-inferior processes are also boney structures and extend generally laterally from vertebrae 12 and allow ligaments, muscles, and tendons to attach to vertebra 12. Vertebral facets join adjacent vertebrae 12 to each other while allowing motion there between by being in sliding contact with corresponding vertebral facets of these adjacent vertebrae 12.

With continuing reference to FIG. 2, during certain types of motion of spine 10 (such as flexing and extending) which can be caused (or resisted) by various muscles, vertebrae 12 tend to rotate relative to each other about axes of rotation generally in intravertebral areas. Intravertebral areas can be adjacent to and posterior to intervertebral disks and substantially anterior to spinous processes and vertebral facets. Since vertebral facets allow vertebrae to articulate about these axes of rotation little or no reactionary forces or moments are generated by healthy spines 10 during ordinary movements.

However, due to various chronic, degenerative, genetic, etc. conditions, spine 10 can be deformed and, in some cases, deformed in significant manners. With reference to FIG. 2, in some instances, spine 10 can be deformed sufficiently that spinal stabilization rod 24, when seated in one particular bone fastener assembly 26, happens to be spaced apart from certain other bone fastener assemblies 26 by some distance. In such cases, spinal stabilization rod 24 can be said to be “proud” of these other bone fastener assemblies 26 by such distances. When spinal stabilization rod 24 is proud of particular bone fastener assemblies 26 but still remains within at least one other bone fastener assembly 26, internal threads of this other bone fastener assembly 26 can allow closure member 28 to be driven down and into that other bone fastener assembly 26, thereby reducing spinal stabilization rod 24 into its intended position in that bone fastener assembly 26.

In some situations, it may be difficult for surgical personnel to reduce spinal stabilization rod 24 into the particular bone fastener assembly 26. For instance, if spinal stabilization rod 24 happens to be proud of its intended position in a particular bone fastener assembly 26 by a distance which places it outside of the particular bone fastener assembly 26, closure member 28 cannot engage the internal threads of the particular bone fastener assembly 26 to reduce spinal stabilization rod 24 into position. Moreover, because spine 10 and the anatomical features coupled thereto may resist movement of spinal stabilization rod 24, it may be difficult for surgical personnel to force spinal stabilization rod 24 into the particular bone fastener assembly 26 absent certain mechanical advantages provided by internal threads of bone fastener assembly 26. Previously, surgical personnel might have had to remove spinal stabilization rods 24, bone fastener assemblies 26, and potentially other portions of spinal stabilization system 22 and attempt to stabilize spine 10 with different spinal stabilization system components or different spinal stabilization systems altogether. Because of the additional surgical steps (and surgical operating time associated there with), the patient can suffer additional trauma to tissues in (or adjacent to) the surgical site, post-operative complications, discomfort, pain, etc.

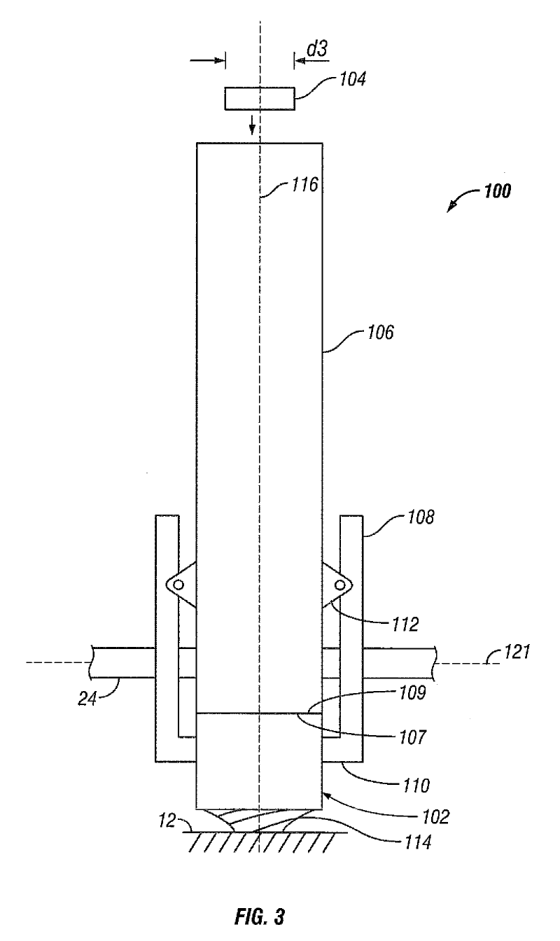

With reference now to FIG. 3, FIG. 3 depicts a side elevation view of one embodiment of rod reduction instrument 100 as seen from a medial lateral direction. FIG. 3 illustrates rod reduction instrument 100 reducing spinal stabilization rod 24 into bone anchor 102. As illustrated in FIG. 3, bone anchor 102 can be anchored to vertebra 12 or some other boney structure. Closure member 104 may be used in conjunction with rod reduction instrument 100 to reduce spinal stabilization rod 24 into bone anchor 102 as is explained in more detail herein. With continuing reference to rod reduction instrument 100, in some embodiments, rod reduction instrument 100 includes instrument body 106, instrument flange 107, a pair of alignment feature actuators 108, alignment features 110, and a pair of actuator gussets 112. Instrument body 106 maybe elongated and define instrument flange 107 at its distal end. Instrument body 106 may define an internal slot (discussed herein in more detail) for accepting closure member 104. Actuator gussets 112 can couple to superior-inferior surfaces of instrument body 106 and pivotably couple alignment feature actuators 108 to instrument body 106. Instrument body 106 may enable surgical personnel to navigate instrument flange 107 to bone anchor 102 (which maybe implanted subpercutaneously) using a number of different approaches. In one embodiment, surgical personnel implant bone anchor 102 and spinal stabilization rod 24 using a posterior approach. Many other approaches are possible and can be used without departing from the scope of the disclosure.

Instrument flange 107 may be shaped, dimensioned, etc. or otherwise adapted to mount rod reduction instrument 100 on bone anchor 102. Actuator gussets 112 of rod reduction instrument 100 may be formed integrally with instrument body 106 or can be formed separately and coupled to instrument body 106 by welding, brazing, using fasteners, etc. Actuator gussets 112 may be positioned toward the distal end of instrument body 106 and can be positioned to provide surgical personnel who might be using rod reduction instrument 100 some mechanical advantage when actuating alignment feature actuators 108. In some embodiments, actuator gussets 112 can be positioned on instrument body 106 sufficiently distant from the distal end of rod reduction instrument 100 so that, when rod reduction instrument 100 is mounted to bone anchor 102, actuator gussets 112 will lie outside of most, if not all, patients' bodies.

Alignment feature actuators 108 can be bars or other generally elongated shapes suitable for pivotably coupling to actuator gussets 112 and for supplying surgical personnel some mechanical advantage when actuating instrument alignment features 110. Alignment feature actuators 108 can extend some distance parallel to longitudinal axis 116 from actuator gussets 112 and toward the proximal end of rod reduction instrument 100. Alignment feature actuators may be sufficiently long so as to allow surgical personnel to grasp the proximal ends of alignment feature actuators 108 when rod reduction instrument 100 is mounted to bone anchor 102. Alignment feature actuators 108 may extend from actuator gussets 112 toward, and beyond, the distal end of rod reduction instrument 100 and, more particularly, beyond instrument flange 107 so that, when rod reduction instrument 100 is mounted on bone anchor 102 (by instrument flange 107), instrument alignment features 110 can engage corresponding alignment features of bone anchor 102. In some embodiments, alignment feature actuators 108 can be biased so that their distal ends are urged in a radial direction toward, or away from instrument body 106, although alignment feature actuators 108 need not be biased in any direction. In some embodiments, alignment feature actuators 108 can be oriented along superior-inferior axis 121 when rod reduction instrument 100 is mounted on bone anchor 102. Alignment feature actuators 108 can be formed integrally with instrument alignment features 110 or they can be formed separately and coupled together by welding, brazing, using fasteners, etc.

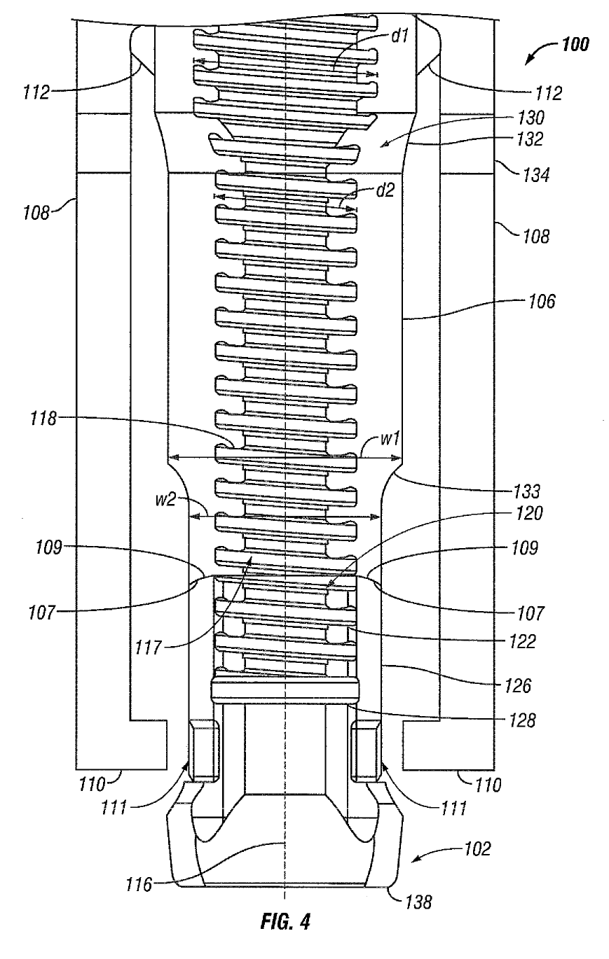

Instrument alignment features 110 can correspond to alignment features of bone anchor 102 (see FIG. 4). Instrument alignment features 111 can couple to, and be actuated by, alignment feature actuators 108. Instrument alignment features 110 can be positioned at the distal end of alignment feature actuators 108 so that, when rod reduction instrument 100 is mounted on bone anchor 102, instrument alignment features 110 are adjacent to bone anchor 102 and, more particularly, adjacent to certain alignment features which bone anchor 102 may have. When surgical personnel actuating alignment feature actuators 108 by grasping their proximal ends (or in any other fashion), alignment feature actuators 108 can cause instrument alignment features 110 to move radially away from instrument body 106 (or toward instrument body 106). Thus, with alignment feature actuators 108 positioned adjacent to superior-inferior surfaces of instrument body 106, alignment feature actuators 108 can urge instrument alignment features 110 in a direction generally parallel to superior-inferior axis 121. In some embodiments, instrument alignment features 110 can have a generally rectangular cross section and can extend sufficiently distant from alignment feature actuator 108 so as to engage certain alignment features of bone anchor 102. Thus, when rod reduction instrument 100 is mounted on bone anchor 102, instrument alignment features 110 can engage, and disengage from, alignment features 10 of bone anchor 102.

At this juncture, it may be useful to discuss aspects of bone anchor 102. Bone anchor 102 can define bone anchor flange 109 on which instrument 100 may be mounted via instrument flange 107. Thus, instrument flange 107 and bone anchor flange 109 may correspond to each other. Bone anchor 102 can further define an internal slot (discussed further herein) for accepting spinal stabilization rod 24. Bone anchor 102 can also include threaded member 114 or other attachment mechanism for attaching bone anchor 102 to vertebrae 12 (or other boney structures).

In operation, surgical personnel may open a surgical site in a patient's body, distract muscles, nerves, arteries, veins, organs, etc. which might be proximal to vertebra 12 (or some other selected boney structure) to which surgical personnel may desire to attach spinal stabilization system 22 (including one or more bone anchors 102). Surgical personnel may then attach bone anchor 102 to vertebrae 12 along with attaching other bone anchors 102 to other vertebrae 12. Spinal stabilization rod 24 may be placed in one or more bone anchors 102 and positioned so that spinal stabilization rod 24 extends in a direction generally parallel to superior-inferior axis 121 to a position proximal to a particular bone anchor 102 of FIG. 3. In some situations, spinal stabilization rod 24 may be formed into a shape which generally follows spine 10 which (because of certain conditions including, but not limited to, those disclosed herein) may, or may not, generally parallel superior-inferior axis 121). In some situations, spinal stabilization rod 24 may be proud of bone anchor 102 by some distance. When such situations, or others, occur surgical personnel can navigate the distal end of rod reduction instrument 100 to a portion of spinal stabilization rod 24 which may be generally proximal to bone anchor 102. Rod reduction instrument 100 can then be navigated in a manner so that a slot (not shown in FIG. 3) of rod reduction instrument 100 and at least extending to the distal end of rod reduction instrument 100 envelopes spinal stabilization rod 24.

Surgical personnel may navigate rod reduction instrument 100 so that instrument flange 107 contacts bone anchor flange 109 of bone anchor 102 with spinal stabilization rod 24 in the internal slot of rod reduction instrument 100. Depending on whether, and in which direction, alignment feature actuators 108 are biased (if it all), surgical personnel may actuate alignment feature actuators 108 to provide clearance between instrument alignment features 110 and bone anchor 102 while instrument flange 107 is brought into contact with bone anchor flange 109. Actuation of alignment feature actuators 108 can be by way of grasping alignment feature actuators 108 at a location proximal from actuator gussets 112 and causing alignment feature actuators 108 to pivot about actuator gussets 112. Rod reduction instrument 100 can be rotated about longitudinal axis 116, or otherwise oriented, to bring instrument alignment features 110 into registry with alignment features of bone anchor 102. Surgical personnel may then release, or actuate, alignment feature actuators 108 to cause instrument alignment features 110 to engage alignment features of bone anchor 102. Instrument alignment features 110, in conjunction with corresponding alignment features of bone anchor 102, can hold rod reduction instrument 100 and bone anchor 102 in fixed relationship to each other as explained herein.

Closure member 104 can be brought into the proximity of the proximal end of rod reduction instrument 100. As discussed previously, instrument body 106 can define a slot at its distal end for accepting spinal stabilization rod 24. This instrument slot (not shown in FIG. 3) may extend through the length of instrument body 106 from instrument flange 107 to the proximal end of rod reduction instrument 100. Such instrument slots can have threads corresponding to the threads of closure member 104. Thus, closure member 104 can be driven (with a screwdriver or other driving instrument) through instrument body 106 along longitudinal axis 116 until it engages rod 24 (within instrument body 106). Because of the threads on closure member 104 and the threads of rod reduction instrument 100, surgical personnel can use a certain amount of mechanical advantage provided by the interaction of the threads of closure member 104 and the threads of instrument body 106 to drive closure member 104 against spinal stabilization rod 24 (despite resistance which might arise from spinal stabilization rod 24). Thus, closure member 104 can be used to reduce spinal stabilization rod 24 through rod reduction instrument 100 between the un-reduced position of spinal stabilization rod 24 and the distal end of rod reduction instrument 100.

With continuing reference to FIG. 3, bone anchor 102 can include an internal slot corresponding to the internal slot of rod reduction instrument 100. Instrument alignment features 110 and alignment features of bone anchor 102 can maintain rod reduction instrument 100 and bone anchor 102 in fixed relationship to each other. In such a fixed relationship to each other, threads in rod reduction instrument 100 and threads in bone anchor 102 can align with each other. Aligned with each other, threads in rod reduction instrument 100 and threads in bone anchor 102 can form a continuous threaded path from the proximal end of rod reduction instrument 100 to a certain location in bone anchor 102. That position in bone anchor 102 can be such that rod reduction instrument 100 and bone anchor 102 can reduce spinal stabilization rod 24 into bone anchor 102. Thus, as closure member 104 drives spinal stabilization rod 24 out of rod reduction instrument 100, threads of closure member 104 can engage threads of bone anchor 102 while disengaging from threads of rod reduction instrument 100. Bone anchor 102 can accept closure member 104 and allow it to continue reducing spinal stabilization rod 24 into bone anchor 102.

At some time, surgical personnel can discontinue driving closure member 104 through either rod reduction instrument 100 or through bone anchor 102 as may be desired. Screwdriver(s), and any other instruments which might be in rod reduction instrument 100, can be withdrawn. Alignment feature actuators 108 can be actuated (or released depending on biasing arrangements associated with alignment feature actuators 108) to disengage instrument alignment features 110 from alignment features of bone anchor 102. Surgical personnel may withdraw rod reduction instrument 100 from bone anchor 102 and the surgical site and close the surgical site if desired. Rod reduction instrument 100 may be used to reduce spinal stabilization rod 24 into additional bone anchors 102 attached to other vertebrae 12 or boney structures. Rod reduction instrument 100 can also be used to reduce additional spinal stabilization rods into other bone anchors which may be positioned on the opposite side of spinous process 16 (see FIG. 2) from spinal stabilization rod 24.

Now with reference to FIG. 4, additional features of rod reduction instrument 100 will be discussed. FIG. 4 depicts a cross sectional view of the distal end of rod reduction instrument 100 and bone anchor 102 as viewed from a medial-lateral direction. FIG. 4 also illustrates instrument body 106, instrument flange 107, alignment feature actuators 108, bone anchor flange 109, instrument alignment features 110, bone anchor alignment features 111, actuator gussets 112, longitudinal axis 116, instrument slot 117, instrument threads 118, bone anchor slot 120, bone anchor threads 122, bone anchor slot walls 126, and hard stop 128 among other features. FIG. 4 illustrates rod reduction instrument 100 mounted on bone anchor 102 by way of instrument flange 107 and bone anchor flange 109. Instrument flange 107 and bone anchor flange 109 can correspond to each other and can form a pair of complimentary convex/concave surfaces as shown by FIG. 4 to allow surgical personnel to center rod reduction instrument 100 on bone anchor 102. FIG. 4 illustrates instrument alignment features 110 disengage from bone anchor alignment features 111 whether by basing action or actuation of alignment feature actuators 108.

As noted previously, FIG. 4 illustrates instrument slot 117, instrument threads 11 8, bone anchor slot 120, and bone anchor threads 122. Instrument body 106 can define instrument slot 117. Instrument slot 117 can extend through instrument body 106 between instrument flange 107 at the distal end of rod reduction instrument 100 and the proximal end of rod reduction instrument 100. Instrument threads 118 can be formed in instrument slot walls 124 and can correspond to threads of closure member 104 (see FIG. 3). Bone anchor 102 can define bone anchor slot 120 and can form bone anchor slot walls 126 of bone anchor slot 120. Bone anchor slot 120 can extend sufficiently into bone anchor 102 to allow spinal stabilization rod 24 (see FIG. 3) to seat in bone anchor 102 while retaining structural integrity of bone anchor 102 during, and after, reduction of spinal stabilization rod 24 into bone anchor 102. Bone anchor 102 can define hard stop 128 associated with bone anchor threads 122 and which can prevent closure member 104 from being driven more than a select distance into bone anchor 102. Bone anchor threads 122 can be formed in bone anchor 102 and can correspond to threads of closure member 104. Thus, instrument threads 118 and bone anchor threads 122 can correspond to each other.

Instrument alignment features 110, bone anchor alignment features 111, instrument threads 118, and bone anchor threads 122 can be positioned and oriented relative to each other so that when instrument alignment features 110 engage bone anchor alignment features 111, the distal end of instrument threads 118 align with the proximal end of bone anchor threads 122. In some embodiments, the transition between instrument threads 118 and bone anchor threads 122 can be sufficiently gradual so that threads of closure member 104 (see FIG. 3) can disengage from one of instrument threads 118 or bone anchor threads 122 and engage the other of instrument threads 118 or bone anchor threads 122 without significant binding, loss of mechanical advantage, etc. while being driven by surgical personnel. Thus, surgical personnel can drive closure member 104 along instrument threads 118 and bone anchor threads 122 as if instrument threads 118 and bone anchor threads 122 form a continuous threaded path even while reducing spinal stabilization rod 24 (see FIG. 3) against reactions likely to be encountered during such surgical techniques.

Rod reduction instrument 100 can define thread transition portion 130 of instrument slot 117 across which instrument threads 118 can transition from diameter d1 to another diameter d2. Diameter d1 of instrument threads 118 toward the proximal end of instrument body 106 from thread transition portion 130 can correspond to, but slightly exceed, diameter d3 (see FIG. 3) of threads of closure member 104. Thus, diameter d1 can allow closure member 104 to be driven along the proximal portion of instrument slot 117 with little, or no resistance, thereby facilitating rapid advancement of closure member 104 toward the distal end of instrument slot 117 where spinal stabilization rod 24 is more likely to be than in the proximal end of instrument slot 117.

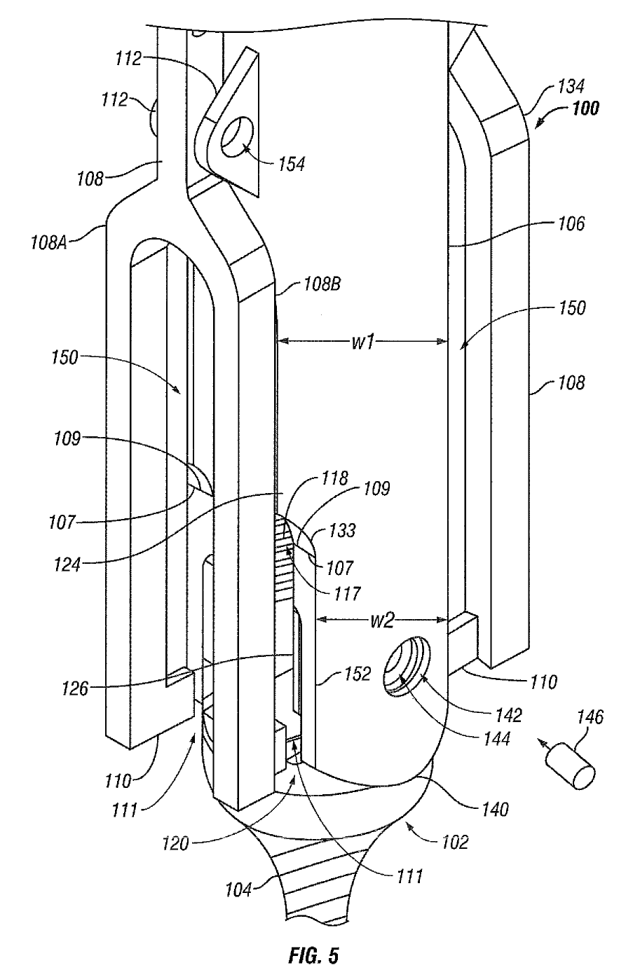

Rod reduction instrument 100 can define body transition portion 132 of instrument body 106 across which instrument body 106 can transition from one thickness to another thickness. Body transition portion 132 can be located at a place along instrument body 106 which is likely to be external to the bodies of most patients when rod reduction instrument 100 is mounted on bone anchor 102. Body transition portion 132 can allow surgical personnel to grasp a more ergonomically dimensioned proximal end of instrument body 106 while minimizing effects on surrounding tissues of the distal end of instrument body 106. Rod reduction instrument 100 can define width transition portion 133 where instrument body 106 transitions from one width w1 to another width w2. Thus, width transition portion 133 can allow the thinner and less intrusive distal end of rod reduction instrument 100 (as compared to the proximal end of instrument body 106) to be navigated within the patients' bodies. Width transition portion 133 can provide clearance between distal portions of alignment feature actuators 108 and instrument slot walls 124 thereby allowing instrument alignment features 110 to engage bone anchor alignment features 111. Alignment feature actuators 108 may define actuator transition portion 134 to be discussed in more detail with reference to FIG. 5.

Bone anchor 102 can include a bone anchor socket (defined by bone anchor gusset 138) at the distal end of bone anchor 102. Such bone anchor sockets can be shaped, dimensioned, etc. to receive and retain a head portion (not shown) of threaded member 114 (see FIG. 3). U.S. patent application Ser. No. 11/959,063, entitled SPINAL STABILIZATION SYSTEMS AND METHODS, and filed on Dec. 18, 2007 discloses spinal stabilization systems and related methods for receiving and retaining bone anchor head portions in bone anchor sockets and is incorporated herein as if set forth in full.

With continuing reference to FIG. 4, the bone anchor socket at the distal end of bone anchor 102 can communicate with bone anchor slot 120. Thus, closure member 104 can come into abutting relationship with head portions of threaded member 114 as surgical personnel drive closure member 104 into bone anchor 102 and more particularly into the distal end of bone anchor slot 120. Because bone anchor 102 can retain the head portion of threaded member 114, the two can hold each other in fixed relationship while provided selected degrees of freedom for threaded member 114 to angulate relative to bone anchor 102.

Bone anchor 102 can define bone anchor gusset 138 which can provide sufficient material about bone anchor socket 136 to withstand reactions between bone anchor 102 and threaded member 114. Bone anchor gusset 138 can also be dimensioned, shaped, etc. to spread forces onto vertebra 12 (or other boney structures to which threaded member 114 can attach bone anchor 102) which might otherwise be concentrated on portions of vertebra 12 should bone anchor 102 come into abutting relationship with vertebra 12. Thus, bone anchor gusset 138 can alleviate various forces which might act on the surface of vertebra 12 while assisting with maintaining the structural integrity and functionality of spinal stabilization system 22 (see FIG. 2).

With reference now to FIG. 5, FIG. 5 depicts a perspective view of one embodiment of rod reduction instrument 100. FIG. 5 also illustrates bone anchor 102, instrument body 106, instrument flange 107, alignment feature actuator 108, bone anchor flange 109, instrument alignment feature 110, bone anchor alignment feature 111, threaded member 114, instrument slot 117, instrument threads 118, bone anchor slot 120, bone anchor threads 122, instrument slot walls 124, bone anchor slot walls 126, width transition portion 133, actuator transition portion 134, instrument tongs 140, second instrument alignment feature 142, and second bone anchor alignment feature 144. FIG. 5 also illustrates widths w1 and w2 of instrument body 106.

FIG. 5 shows instrument body 106 partially enveloping bone anchor 102. More particularly, instrument body 106 can include instrument tongs 140 at the distal end of instrument body 106. Width transition portion 133 can couple instrument tongs 140 to the remainder of instrument body 106. Instrument tongs 140 can slidably engage a portion of bone anchor slot walls 126. By slidably engaging bone anchor slot walls 126 (shown in FIG. 5 behind tong edge 152), instrument tongs 140 can provide alignment between rod reduction instrument 100 and bone anchor 102 relative to longitudinal axis 116 (see FIG. 4). Second instrument alignment feature 142 and second bone anchor alignment feature 144 can be positioned on bone anchor 102 and instrument tongs 140, respectively, to register with each other. Second instrument alignment feature 142 and second bone anchor alignment feature 144 can be dimensioned, shaped, etc. to correspond to each other. Thus, alignment locking pin 146 can be inserted through second instrument alignment feature 142 and through second bone anchor alignment feature 144 to hold rod reduction instrument 100 and bone anchor 102 in fixed relationship to each other when rod reduction instrument 100 is mounted on bone anchor 102. In such fixed relationship, instrument threads 118 and bone anchor threads 122 (also shown in FIG. 4) can be aligned with each other to provide a substantially continuous thread along instrument slot 117 and bone anchor slot 120.

FIG. 5 also illustrates actuator transition portion 134 and actuator slots 150. Actuator slots 150 can be shaped and dimensioned to accept spinal stabilization rod 24 (see FIG. 2). Actuator slots 150 can be defined by respective pairs of actuator arms 108A and 108B. Together, actuator arms 108A and 108B and actuator transition portion 134 can lend actuator a “wish bone” shape when viewed from a superior-inferior direction. Actuator slots 150 can correspond to instrument slot 117 and to bone anchor slot 120. Thus, as surgical personnel navigate rod reduction instrument 100 to bone anchor 102, alignment feature actuators 108 can allow instrument slot 117 to accept spinal stabilization rod 24.

As illustrated by FIG. 5 instrument alignment features 110 can be held in spaced apart relationship from bone anchor 102 by actuation of alignment feature actuators 108 when rod reduction instrument 100 is mounted on bone anchor 102. When instrument alignment features 110 register with bone anchor alignment features 111, alignment feature actuators 108 can urge instrument alignment features 110 into bone anchor alignment features 111. Instrument alignment features 110 and bone anchor alignment features 111 can therefore hold rod reduction instrument 100 and bone anchor 102 in fixed relationship with instrument threads 118 and bone anchor threads 122 in alignment. Accordingly, closure member 104 (see FIG. 3) can be driven along instrument slot 117 and bone anchor slot 120 substantially without binding or losing mechanical advantage as closure member 104 translates between instrument slot 117 and bone anchor slot 120.

FIG. 5 also illustrates that actuator gussets 112 can define hinge apertures 154. Hinge apertures 154 can correspond to apertures defined by alignment feature actuators 108. Pins, or other members, can be inserted through hinge apertures 154 and their corresponding apertures defined by alignment feature actuators 108 to pivotably couple alignment feature actuators 108 to instrument body 106. Thus, surgical personnel may grasp proximal portions of alignment feature actuators 108 to actuate alignment feature actuators 108 and translate instrument alignment features 110 relative to bone anchor alignment features 111. More particularly, alignment feature actuators 108 can translate instrument alignment features 110 in a radial direction (relative to longitudinal axis 116) toward, and away from, instrument body 106.

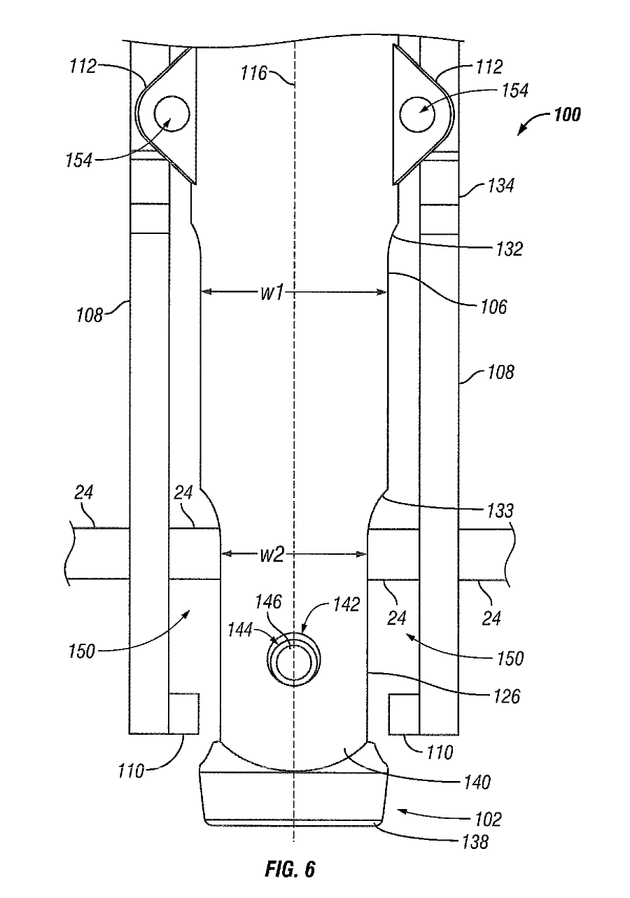

With reference now to FIG. 6, FIG. 6 depicts a side elevation view of one embodiment of rod reduction instrument 100. FIG. 6 illustrates bone anchor 102, instrument body 106, alignment feature actuator 108, instrument alignment feature 110, actuator gussets 112, longitudinal axis 116, bone anchor slot walls 126, body transition portion 132, width transition portion 133, actuator transition portion 134, bone anchor gusset 138, instrument tongs 140, second instrument alignment feature 142, bone anchor alignment feature 144, alignment locking pin 146, actuator slots 150, and instrument body widths w1 and w2. FIG. 6 also shows that alignment feature actuators 108 can be on the superior and inferior sides of rod reduction instrument 100 (when rod reduction instrument 100 is mounted on bone anchor 102 with spinal stabilization rod 24 in instrument slot 117 and oriented along a superior-inferior direction. Thus, alignment feature actuators 108 can actuate instrument alignment features 110 in a superior-inferior direction, thereby allowing instrument alignment features 110 to translate relative to spine 10 along spine 10 where surgical personnel may have opened an incision for attaching spinal stabilization rod 24 to vertebrae 12.

Actuator gussets 112 may be positioned on superior and inferior surfaces of instrument body 106 and can form part of actuator hinge 154. Second instrument alignment feature 142, second bone anchor alignment feature 144, and alignment locking pin 146 can be located on medial/lateral surfaces of instrument body 106. Being placed on medial/lateral surfaces of instrument body 106 and bone anchor 102, second instrument alignment feature 142 and second bone anchor alignment feature 144 can be positioned sufficiently distant from alignment feature actuators 108 and instrument slots 117 so that actuation of instrument alignment features 110 and reduction of spinal stabilization rod 24 is not affected by the location of second instrument alignment feature 142, second bone anchor alignment feature 144, or alignment locking pin 146 other than assisting in holding rod reduction instrument 100 and bone anchor 102 in fixed relationship to each other.

As discussed previously, rod reduction instrument 100 may be navigated to bone anchor 102 and mounted thereon. Since it is possible that second instrument alignment feature 142 and second bone anchor alignment feature 144 may be out of registration with each other when rod reduction instrument 100 is initially mounted on bone anchor 102, it may be necessary to bring second instrument alignment feature 142 and second bone anchor alignment feature 144 to place alignment locking pin 146 therein. To do so, surgical personnel can rotate rod reduction instrument 100 until second instrument alignment feature 142 registers with second bone anchor alignment feature 144. Alignment locking pin 146 can be inserted into second instrument alignment feature 142 and second bone anchor alignment feature 144. Second instrument alignment feature 142, second bone anchor alignment feature 144, and alignment locking pin 146 can include provisions to releasably hold alignment locking pin 146 in second instrument alignment feature 142 and second bone anchor alignment feature 144. Such provisions can include detents, bayonet members, etc.

In some embodiments, alignment locking pin 146 can include a head or other feature to prevent alignment locking pin 146 from translating through second instrument alignment feature 142 and second bone anchor alignment feature 144. After, spinal stabilization rod 24 is reduced, as discussed herein, alignment locking pin 146 can be removed from second instrument alignment feature 142 and second bone anchor alignment feature 144 to allow rod reduction instrument 100 to be withdrawn from bone anchor 102 (with appropriate actuation of instrument alignment features 110).

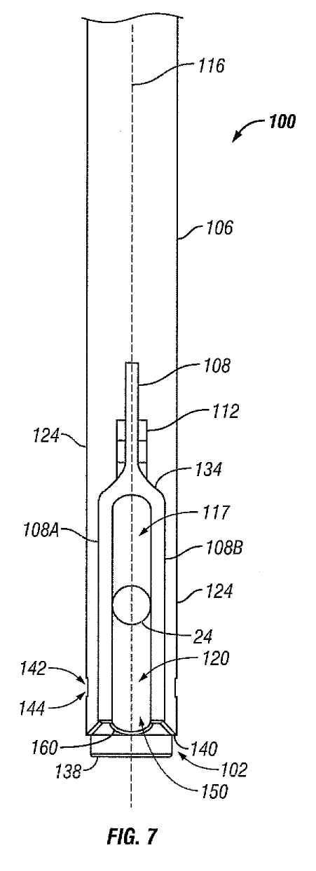

With reference now to FIG. 7, FIG. 7 depicts a side elevation view of rod reduction instrument 100 as seen from a superior-inferior direction. FIG. 7 also illustrates instrument body 106, alignment feature alignment feature actuator 108, actuator arm 108A, actuator arm 108B, actuator gusset 112, longitudinal axis 116, instrument slot 117, bone anchor slot 120, instrument slot walls 124, actuator transition portion 134, bone anchor gusset 138, instrument tongs 140, second instrument alignment feature 142, second bone anchor alignment feature 144, actuator slot 150, and spinal stabilization rod seat 160. In some embodiments, alignment feature actuator 108 can include actuator arms 108A and 108B which define actuator slot 150. Actuator slot 150 can correspond to instrument slot 117 and bone anchor slot 120. Thus, together, actuator slot 150, instrument slot 117, and bone anchor slot 120 can allow spinal stabilization rod 24 to be received by rod reduction instrument 100 (despite potentially be proud of bone anchor 102 by some distance) and reduced through instrument slot 117 into bone anchor slot 120. As spinal stabilization rod 24 is reduced into bone anchor slot 120, at some point spinal stabilization rod 24 can come into contact with spinal stabilization rod seat 160. Since spinal stabilization rod seat 160 can be shaped, dimensioned, etc. to correspond to spinal stabilization rod 24, spinal stabilization rod seat 160 (in conjunction with closure member 104) can hold spinal stabilization rod 24 in fixed relationship to bone anchor 102 and spine 10 (see FIG.2). Spinal stabilization rod 24 can be grasp by surgical personnel and maneuvered to other bone anchors 102 attached to other vertebra 12. Spinal stabilization rod 24 can be reduced into these other bone anchors to treat certain conditions affecting spine 10.

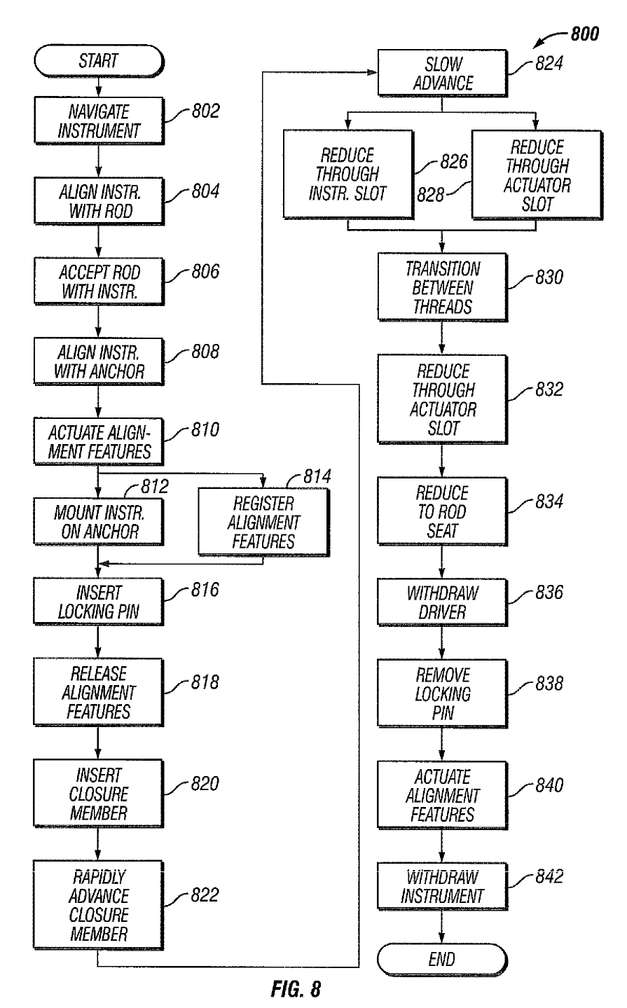

With reference now to FIG. 8, method 800 of reducing spinal stabilization rod 24 is illustrated. Method 800 can include various steps such as opening a surgical site and preparing an area of spine 10 (see FIG. 2) for implanting of spinal stabilization system 22 into a patient's body. Attaching spinal stabilization system 22 to spine 10 can include attaching one or more bone anchors 102 to vertebrae 12 and reducing spinal stabilization rod 24 into such bone anchors 102. In method 800, rod reduction instrument 100 can be navigated to the surgical site and, more particularly, to the proximity of a particular bone anchor 102 and spinal stabilization rod 24 at step 802. Surgical personnel can align instrument slot 117 (and actuator slot 150) with spinal stabilization rod 24 at step 804. Rod reduction instrument 100 can then be navigated so that instrument slot 117 (and actuator slot 150) accept spinal stabilization rod 24 at step 806. At step 808, surgical personnel can (with spinal stabilization rod 24 in instrument slot 117 and actuator slot 150) align rod reduction instrument 100 with bone anchor 102 along longitudinal axis 116. Surgical personnel can actuate instrument alignment features 110 using alignment feature actuators 108 to provide clearance between instrument alignment features 110 and bone anchor 102 during various steps of method 800. Actuation of instrument alignment features 110 can be by way of grasping the proximal ends of alignment feature actuators 108 and causing alignment feature actuators 108 to pivot about pivot gussets 112.

At some time, rod reduction instrument 100 can be mounted to bone anchor 102 at step 812. Rod reduction instrument 100 can be rotated to bring instrument alignment features 110 into registration with bone anchor alignment features 111 at step 814. In some embodiments, second instrument alignment features 142 and bone anchor alignment features 144 can be brought into registration at step 814. If desired, alignment locking pin 146 can be inserted into second instrument alignment feature 142 and second bone anchor alignment feature 144 at step 816. Surgical personnel may release instrument alignment feature actuator 108 to cause instrument alignment feature 110 to engage bone anchor alignment features 111 at step 818. Thus, rod reduction instrument 100 can be mounted to bone anchor 102 with instrument threads 118 and bone anchor threads 122 aligned with each other and forming one substantially continuous threaded path along instrument slot 117 and bone anchor slot 120.

Surgical personnel can insert closure member 104 into the distal end of instrument slot 117 at step 820 and begin driving it along instrument slot 117. While closure member 104 is encountering instrument threads 118 of diameter d2 (see FIG. 4), surgical personnel can drive closure member 104 relatively rapidly because diameter d2 can be selected so that instrument threads 118 (of diameter d2) provide little or no resistance to the advancement of closure member 104. Thus, at step 822, surgical personnel can advance closure member 104 relatively rapidly thereby consuming minimal surgical time advancing closure member 104 while closure member 104 is not in abutting relationship with spinal stabilization rod 24. As closure member 104 engages instrument threads 118 in thread transition portion 130 of instrument slot 117, the decrease of thread diameters from diameter d2 to diameter d1 can cause instrument threads 118 to cause some resistance to the advancement of closure member 104. Thus, at step 824, the advancement of closure member 104 can slow.

At some time, closure member 104 can be advanced into abutting relationship with spinal stabilization rod 24. Surgical personnel, at step 826, can drive closure member 104 to reduce spinal stabilization rod 24 through a portion of instrument slot 117. In embodiments involving actuator slots 150, method 800 can include reducing spinal stabilization rod 24 through actuator slots 150 at step 828. As spinal stabilization rod 24 is reduced through rod reduction instrument 100, rod reduction instrument 100 and bone anchor 102 can be placed in tension. The tension can arise from the reactions between closure member 104 and spinal stabilization rod 24 as closure member 104 is driven through instrument slot 117 (driving spinal stabilization rod 24 before it). Such tensile forces can cause instrument alignment features of some rod reduction instruments to roll off the edges of corresponding anchor alignment features. Surgical personnel may hold, lock, or otherwise restrain alignment feature actuators 108 to maintain instrument alignment features 110 in engagement with anchor alignment features 111 as spinal stabilization rod 24 is reduced at steps 826 and 828 (and steps 830 and 834).

At some time, leading threads of closure member 104 can encounter bone actuator threads 122 as surgical personnel continue driving closure member 104 toward spinal stabilization rod seat 160 (see FIG. 7) of bone anchor 102. Closure member 104 can therefore transition from engaging instrument threads 118 to engaging bone anchor threads 122 at step 830. As closure member 104 transitions to engaging bone anchor threads 122, closure member 104 can experience little, or no, binding or loss of mechanical advantage because instrument threads 118 and bone anchor threads 122 can form a substantially continuous threaded path across instrument flange 107 and bone anchor flange 109.

At step 832, surgical personnel can continue driving closure member 104 toward spinal stabilization rod seat 160 thereby reducing spinal stabilization rod 24 through bone anchor slot 120. Spinal stabilization rod 24 can come into abutting relationship with spinal stabilization rod seat 160 thereby becoming seated on spinal stabilization rod seat 160 at step 834. When satisfied with reduction of spinal stabilization rod 24 into bone anchor 102, surgical personnel can withdraw from instrument slot 117 any instrument (such as a screwdriver) which they may have used to drive spinal stabilization rod 24 at step 836. Surgical personnel can remove alignment locking pin 146 from second instrument alignment feature 142 and second bone anchor alignment feature 144 at step 838. Surgical personnel may actuate instrument alignment features 110 thereby causing instrument alignment features 110 to disengage from bone anchor alignment features 111 while translating radially away from the same at step 640. Rod reduction instrument 100 may be withdrawn from bone anchor 102 (and the surgical site if desired) at step 842. In some embodiments, surgical personnel can use rod reduction instrument 100 to reduce rod 24 into additional bone anchors 102 or surgical personnel can reduce additional spinal stabilization rods into other bone anchors 102. When surgical personnel have finished using rod reduction instrument 100 to reduce spinal stabilization rods 24, surgical personnel can withdraw rod reduction instrument 100 from the surgical site and close the same.

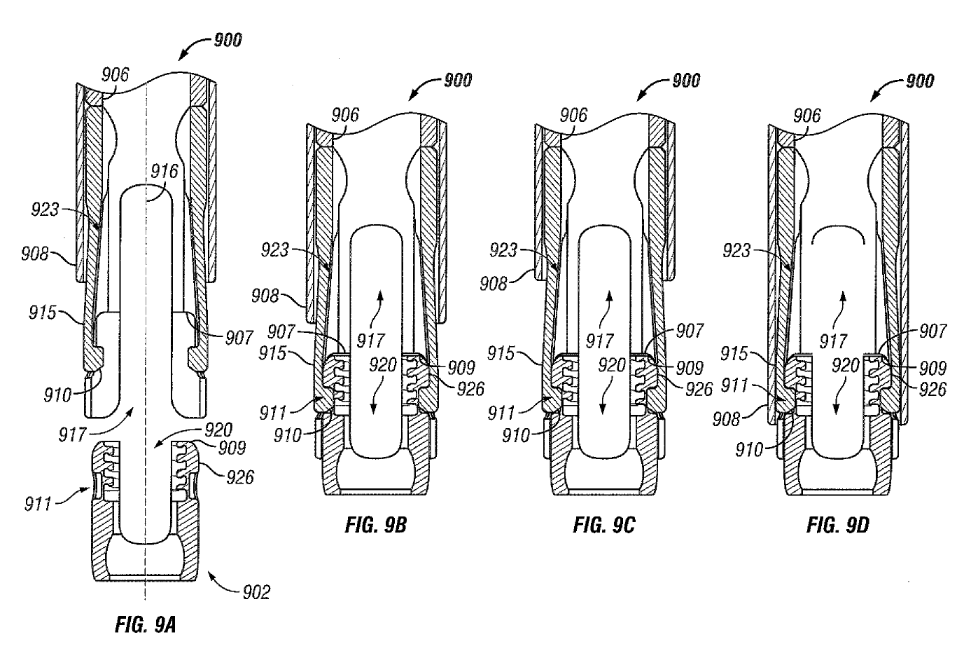

With reference to FIGS. 9A-9D, in which one embodiment of rod reduction instrument 900 and one embodiment of bone anchor 902 are illustrated. FIG. 9A shows bone anchor 902 adjacent to rod reduction instrument 900 but un-captured thereby, while FIGS. 9B-9D show instrument 100 having captured bone anchor 902.

With reference now to FIG. 9A one embodiment of rod reduction instrument 900 for reducing spinal stabilization rods 24 into bone anchors 902 is illustrated. In some embodiments, rod reduction instrument 900 can be adapted to maintain instrument 900 and bone anchor 902 in alignment as rod reduction instrument 900 and bone anchor 902 are placed in tension as closure member 104 (see FIG. 3) engages spinal stabilization rod 24. More particularly, bone anchor 902 can include anchor flange 909 and anchor side walls 926 and can define anchor slot 920 and anchor alignment features 911. Rod reduction instrument 900 can include instrument body 906, instrument flange 907, instrument sleeve 908, instrument alignment features 910, and instrument resilient fingers 915 and can define instrument slot 917 and instrument reduced cross sectional portion 923. Rod reduction instrument 900 can have longitudinal axis 916 along which a linear actuator (not shown) can actuate instrument body 906 (and instrument resilient fingers 915) in a direction along longitudinal axis 916. The instrument actuator could be any type of linear actuator, although, in some embodiments, the actuator can be a push-to-lock-push-to-release actuator.

Instrument resilient fingers 915 can extend from the distal end of instrument body 906 and can be biased against instrument reduced cross sectional portion 923. Instrument reduced cross sectional portion 923 and instrument resilient fingers 915 can be shaped and dimensioned to correspond to each other so that together they generally correspond to the overall cross sectional shape and dimensions of the remainder of instrument body 906.

With reference now to FIGS. 9B-9D, rod reduction instrument 900 can include three positions defined with reference to the position of instrument body 906 (and instrument resilient fingers 915) relative to instrument sleeve 908. More particularly, FIG. 9B shows instrument 100 in a position in which instrument resilient fingers 915 are locked in a generally distal position relative to instrument resilient fingers 915. The position illustrated by FIG. 9B can correspond to the position where a push-to-lock-push-to-release actuator is in its unlocked position. In the position of FIG. 9B, instrument sleeves 908 do not significantly engage instrument resilient fingers 915. Thus, instrument resilient fingers 915 can move in a radial direction relative to instrument body 906.

FIG. 9C illustrates instrument resilient fingers 915 in a slightly more distal position relative to instrument sleeve 908 in which the push-to-lock-push-to-release actuator has been pushed in a distal direction to move it away from its unlocked position. In FIG. 9C, instrument sleeves 908 are shown as not significantly engaging instrument resilient fingers 915. Thus, instrument resilient fingers 915 can move in a radial direction relative to instrument body 906.

In FIG. 9D, rod reduction instrument 900 is shown as being in a position in which instrument resilient fingers 915 have been withdrawn in a proximal direction toward, and into, instrument sleeve 908. Instrument sleeve 908, in FIG. 9D, engage instrument resilient fingers 915, thereby holding instrument resilient fingers 915 against instrument body 906 with instrument alignment features 910 engaging anchor alignment features 911. The push-to-lock-push-to-release actuator can lock rod reduction instrument 900 in the position shown in FIG. 9D. Thus, instrument resilient fingers 915 can be restrained from moving in a radial direction relative to instrument body 906. Pushing the push-to-lock-push-to-release actuator again can cause rod reduction instrument 900 to return to the position illustrated by FIG. 9B.

In operation, surgical personnel can navigate instrument 900 to bone anchor 902 as illustrated in FIG. 9A. Surgical personnel can advance instrument 900 (in its position as shown in FIG. 9A and FIG. 9B) along longitudinal axis 916. Instrument resilient fingers 915 can be free to move radially relative to instrument body 906. As surgical personnel do so, rod reduction instrument 900 can capture bone anchor 902, instrument resilient fingers 915 can slidably engage anchor side walls 926. As instrument resilient fingers 915 advance along anchor side walls 926, instrument alignment features 910 can engage anchor alignment features 911.

Surgical personnel can push the push-to-lock-push-to-release actuator to actuate instrument body 906 relative to instrument sleeve 908. As the push-to-lock-push-to-release actuator is pushed, instrument 900 can move to the position illustrated by FIG. 9C. At the position shown in FIG. 9C, instrument resilient fingers 915 can remain engaged with anchor alignment features 915 even though instrument sleeve 908 is spaced apart from instrument resilient fingers 915. Surgical personnel can release the push-to-lock-push-to-release actuator so that instrument 900 moves to the position illustrated by FIG. 9D. In that position, instrument resilient fingers 915 can withdraw in a proximal direction relative to instrument sleeve 908. As instrument resilient fingers 915 withdraw into instrument sleeve 908, instrument sleeve 908 can engage instrument resilient fingers 915, thereby holding them against instrument body 906 and in engagement with anchor alignment features 911.

Since the push-to-lock-push-to-release actuator can lock with instrument 900 in the position illustrated by FIG. 9D, instrument sleeve 908 can lock instrument alignment features 910 and anchor alignment features 911 in engagement with each other. Surgical personnel can utilize closure member 104 to drive spinal stabilization rod 24 through instrument slot 917 and anchor slot 920. With instrument 900 locked in the position illustrated by FIG. 9D, instrument sleeve 908 can resist forces (arising from the reduction of spinal stabilization rod) which might otherwise tend to cause instrument alignment features 910 to disengage from anchor alignment features 911. Thus, rod reduction instrument 900 can be adapted to maintain instrument 900 and bone anchor 902 in alignment as rod reduction instrument 900 and bone anchor 902 are placed in tension as closure member 104 (see FIG. 3) engages spinal stabilization rod 24.

In the foregoing specification, specific embodiments have been described with reference to the accompanying drawings. However, as one skilled in the art can appreciate, embodiments of the spinal stabilization rod reduction instrument and bone anchor disclosed herein can be modified or otherwise implemented in many ways without departing from the spirit and scope of the disclosure. Accordingly, this description is to be construed as illustrative only and is for the purpose of teaching those skilled in the art the manner of making and using embodiments of spinal stabilization rod reduction instruments and bone anchors. It is to be understood that the embodiments shown and described herein are to be taken as exemplary. Equivalent elements or materials may be substituted for those illustrated and described herein. Moreover, certain features of the disclosure may be utilized independently of the use of other features, all as would be apparent to one skilled in the art after having the benefit of this description of the disclosure.

1. An instrument for reducing a spinal stabilization rod into a bone anchor using a closure member, the bone anchor defining a bone anchor slot for receiving the spinal stabilization rod and the closure member, the bone anchor including a bone anchor alignment feature and a bone anchor slot wall which defines a bone anchor thread, the instrument comprising:
a body having a proximal end and a distal end;
an instrument alignment feature corresponding to the bone anchor alignment feature;
a flange at the distal end of the instrument and being adapted for mounting the instrument on the bone anchor, the instrument defining an instrument slot extending between the proximal end of the instrument and the instrument flange and corresponding to the bone anchor slot; and
an instrument slot wall which defines a thread, the instrument alignment feature being adapted to align the bone anchor and instrument threads in conjunction with the bone anchor alignment feature to form a substantially continuous thread when the instrument is mounted on the bone anchor.

2. The instrument of claim 1 further comprising a thread transition portion of the instrument at which the instrument threads transitions from one diameter to another diameter.

3. The instrument of claim 1 further comprising an alignment feature actuator coupled to the instrument alignment feature to actuate the instrument alignment feature.

4. The instrument of claim 3 wherein the alignment feature actuator defines an actuator slot corresponding to the instrument slot.

5. The instrument of claim 3 wherein the alignment feature actuator is biased to urge the instrument alignment feature toward the bone anchor alignment feature when the instrument is mounted on the bone anchor.

6. The instrument of claim 3 wherein the alignment feature actuator is a sleeve.

7. The instrument of claim 3 wherein the instrument alignment feature is oriented in a radial direction relative to the instrument.

8. The instrument of claim 1 further comprising an anti-splay feature of the instrument thread.

9. The instrument of claim 1 wherein the instrument alignment feature is a pin.

10. A method comprising:
receiving a spinal stabilization rod in an instrument slot of an instrument;
receiving a closure member in the instrument slot;
mounting the instrument on a bone anchor which is adapted for receiving the spinal stabilization rod and the closure member;
aligning an instrument thread of the instrument slot with a bone anchor thread of the bone anchor so that the instrument thread and the bone anchor thread form a substantially continuous thread; and
driving the closure member along the substantially continuous thread to urge the spinal stabilization rod into the bone anchor.

11. The method of claim 10 further comprising driving the closure member along a thread transition portion of the instrument thread at which the instrument threads transitions from one diameter to another diameter.

12. The method of claim 10 wherein the aligning the instrument and bone anchor threads further comprises actuating an alignment feature actuator of the instrument to urge an instrument alignment feature of the instrument away from the bone anchor.

13. The method of claim 12 wherein the actuating the alignment feature actuator further comprises slidably engaging a member coupled to the instrument alignment feature with the alignment feature actuator.

14. The method of claim 12 wherein the actuating the alignment feature actuator is in a radial direction relative to the instrument.

15. An instrument for reducing a spinal stabilization rod into a bone anchor using a closure member, the bone anchor defining a bone anchor slot for receiving the spinal stabilization rod and the closure member, the bone anchor including a bone anchor alignment feature and a bone anchor slot wall which defines a bone anchor thread, the instrument comprising:
a body having a proximal end and a distal end;
an instrument alignment feature corresponding to the bone anchor alignment feature, being oriented in a radial direction relative to the instrument, and being biased toward the bone anchor alignment feature when the instrument is mounted on the bone anchor;
a flange at the distal end of the instrument and being adapted for mounting the instrument on the bone anchor, the instrument defining an instrument slot extending between the proximal end of the instrument and the instrument flange and corresponding to the bone anchor slot;
an instrument slot wall which defines a thread, the instrument alignment feature being adapted to align the bone anchor and instrument threads in conjunction with the bone anchor alignment feature to form a substantially continuous thread when the instrument is mounted on the bone anchor; and
an alignment feature actuator coupled to the instrument alignment feature to actuate the instrument alignment feature and being a sleeve.
